(19) United States
(12) Patent Application Publication
(10) Pub. No.: US 2021/0252045 A1
(43) Pub. Date: Aug. 19, 2021

(54) Method for inactivating and removing disease-related levels of sulfane sulfur from affected biological tissues with special relevance to Covid-19 virus infection

(71) Applicant: John I. Toohey, Elgin (CA)

(72) Inventor: John I. Toohey, Elgin (CA)

(21) Appl. No.: 17/118,841

(22) Filed: Dec. 11, 2020

Related U.S. Application Data
(60) Provisional application No. 62/974,552, filed on Dec. 11, 2019.

(51) Int. Cl. A61K 33/04; A61P 35/00; A61P 31/16; A01N 59/02

(52) U.S. Cl. CPC A61K 33/04; A01N 59/02; A61P 31/16; A61P 35/00

(57) ABSTRACT

Disease states such as cancer or virus infection appear to be dependent on supra-physiological levels of sulfane sulfur. This form of sulfur (sulfane sulfur, zero valent sulfur) has been shown to have numerous biological functions as a growth factor, metabolic regulator, and redox enhancer. It is generated in vivo from the sulfur-containing amino acids methionine and cysteine mainly via the polyamine pathway coupled to cysteine transamination with macrophages acting as an important source. Sulfane sulfur normally binds loosely to sulfur in proteins but it binds tightly to certain agents that have unused electron pairs such as sulfites, nitriles, or phophines. This provides a mechanism for binding and removing disease-related sulfane sulfur. The invention is particularly applicable to Covid-19 because this virus creates a “perfect storm” in which macrophages carry the virus docking site (ACE) and, at the same time, generate the sulfane sulfur required for invasion of the virus. Based on experience with the corona virus of the common cold, it is anticipated that Covid 19 infection process will be abrogated if the sulfane sulfur binding agent is applied within the first few days.

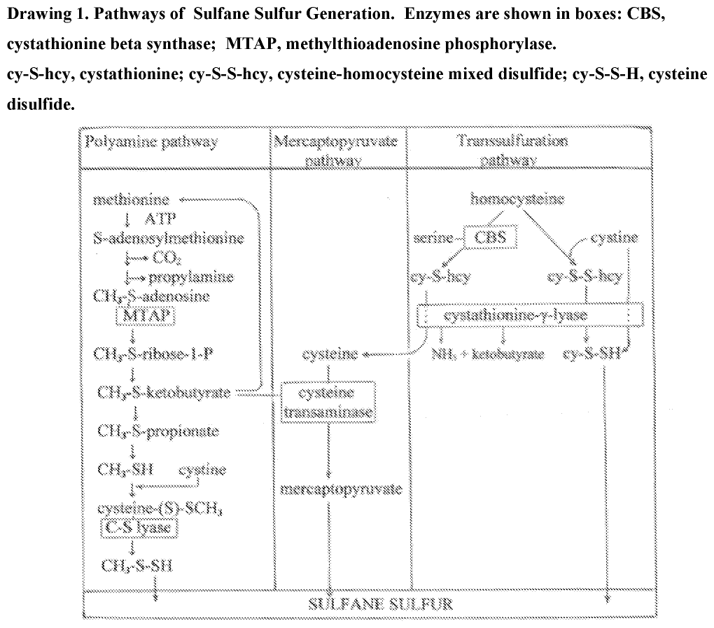

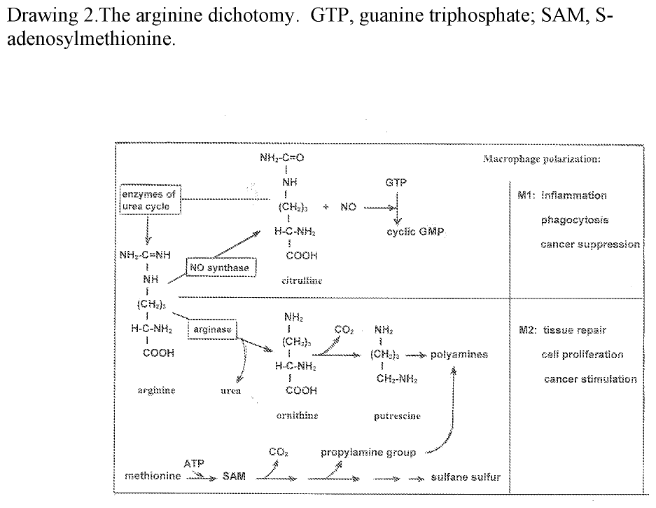

### CROSS REFERENCE TO A RELATED APPLICATION

Application No. 62/974,552

Filing date Dec. 11, 2019

### BACKGROUND OF THE INVENTION, CATEGORY AND PRIOR ART

In recent years, sulfur in the form of “sulfane sulfur” has been found to have regulatory functions in diverse biological processes. Consequently, numerous methods of delivering sulfane sulfur to biological systems have been invented and patented (e.g. AU 2015210403B2). The scientific literature became confused because hydrogen sulfide exposed to air at neutral pH autoxidizes readily to sulfane sulfur (H₂S+O₂→S+H₂O). Therefore, all solutions of hydrosulfide contain sulfane sufur and mimic the effect of pure preprations of it. For that reason there are also patents on agents for delivering hydrogen sulfide pharmaceutically (e.g. JP2017186362A). However, it is apparent to this inventor that certain pathological conditions are caused or stimulated by an excess of sulfane sulfur. According to the present invention there is provided a method of removing disease-related amounts of sulfane sulfur by administration a sufane sulfur-binding agent. This approach is an alternative to strategies such as limiting dietary sulfur intake, destroying methionine with recombinant enzyme, or blocking metabolic pathways (all of which have intolerable side effects). In this application, the following details are described: the discovery of the biological function of sulfane sulfur, the nature of sulfane sulfur, the generation of sulfane sulfur in vivo, the role of macrophages in providing sulfane sulfur, its relationship to disease states, and agents that trap and remove this sulfur.

### 1. Discovery; Murine Lymphoma Cell Lines Dependent on Sulfane Sulfur In Vitro

In the 1950's, the murine lymphoma cells lines, L1210 and P388, were crucial for the screening and pre-clinical testing of the early anti-cancer drugs. At that time, these cells lines could not be cultured in vitro and had to be maintained and used as ascites tumors in live mice. In the 1970's at UCLA, I discovered that these cells could be cultured in vitro in media containing certain sulfur compounds. For routine maintenance of the cells in continuous cell culture, the disulfide, cysteine(S)—S—CH₃ was used (1). At one time, it was thought that the essential factor was the CH₃—S group since, at that time, that group was a popular means of modifying proteins (2). However, the disulfide cysteine(S)—S—CH₃, when degraded metabolically, releases the persulfide, CH₃—S—SH, the outer sulfur of which is a sulfane sulfur. Eventually, it turned out that the sulfane sulfur atom was the essential factor and other systems that generate sulfane sulfur could support growth of the cells in culture (cystine plus pyridoxal, cystamine plus diamine oxidase, mercaptoethanol dislfide plus alcohol dehydrogenase, and sulfide-treated proteins) (3). The sulfane sulfur-dependent cells were found to have complete absence of the enzyme methylthioadenosine phosphorylase (MTAP) (4)—an enzyme in the polyamine pathway which can generate sulfane sulfur as described below.

More recently, the growth factor effect of sulfane sulfur has expanded to include many regulatory functions in biological systems. It modifies bases in mRNA thereby regulating proteins synthesis, it modifies cysteine residues of many proteins regulating their activity; it bonds to glutathione increasing its reductive capacity by at least 20-fold; and it is the source of sulfur for synthesis of iron-sulfur clusters, biotin, lipoic acid, and molybdenum cofactor (5). Many of the physiological effects involve cell proliferation or tissue repair—harkening back to the growth factor effect. There are hundreds of papers—as an example, see (6). The effective concentration range of sulfane sulfur in biological systems is in the nanomolar range and very narrow; i.e. concentrations below or above the optimal range are ineffective or toxic.

Early in my study of sulfane sulfur-dependent cells, there was an additional finding of special interest—nurse cells. When macrophages were isolated from mice by peritoneal lavage and plated on cell culture dishes, they attached and remained viable. The lymphoma cells (L1210, P388) as free-floating suspensions over these macrophages, proliferated profusely in the absence of supplemental sulfane sulfur (7). This indicates that macrophages can generate the growth factor and provide it to other cells. The nurse cell effect of macrophages has expanded and evolved with time into the field of tumor-associated macrophages and is discussed in more detail below.

### 2. The Nature of Sulfane Sulfur

Sulfane sulfur is a form of the sulfur atom in the oxidation state zero with 6 electrons in the valence shell represented as S⁰ or :S: (where represents an electron pair (5). In the scientific literature it has been called zero valent sulfur, elemental sulfur, thiosulfoxide sulfur, persulfide sulfur, polysulfide sulfur, and sulfur-bonded sulfur. Sulfane sulfur has a strong tendency to acquire two more electrons to meet the Lewis 8-electron rule. In biological systems, it usually fills the 8-electron complement by “borrowing” two unused electrons from other sulfur atoms forming relatively weak sulfur-to-sulfur bonds. It bonds in this way to:
organic sulfides such as cysteine or glutathione to give persulfides:

R:S:H+S⁰→R:S(S):H↔R:S:S:H Eq. 1
or organic disulfides to give trisulfides

R:S:S:R+S⁰→R:S(S):S:R↔R:S:S:S:R Eq. 2

Since the S—S bonding in these products is weak, the sulfane sulfur atom can move relatively easily from one substrate sulfur atom to another forming the basis for the regulatory functions. When the cysteine moiety accepting the sulfane sulfur is in a protein, the attachment of a sulfur atom modifies the protein and its activity. Specialized sulfane sulfur-carrying proteins have evolved to transport and deliver the sulfur to appropriate sites. These carriers are highly conserved in all forms of life and contain the motif CxxCxxxC (5).

In contrast to the weak bonding of sulfane sulfur to the organic sulfides and disulfides as described above, there is much more stable chemical bonding to certain electron donors When the sulfane sulfur atom bonds to one of these electron donors, it cannot be easily released and, if this happens in animals, the bonded unit is excreted in the urine. Bonding of this type occurs with sulfite ion, cyanide ion, and phosphines, as described in Section 6.

### 3. Generation of Sulfane Sulfur In Vivo

In animals, sulfane sulfur is derived metabolically from the sulfur amino acids, cysteine and methionine. Three metabolic pathways have been advanced as sources of sulfane sulfur (as shown in Drawing 1):
the cysteine transaminase-mercaptopyruvate sulfur transferase pathway,
the transsulfuration pathway, and
the polyamine pathway.

Transamination or deamination of cysteine yields mercaptopyruvate in which the sulfur atom is activated by the carbonyl group (5). The sulfur atom is transferred to a specific carrier —“mercaptopyruvate sulfur transferase” for transport and delivery to sulfur atoms at receptor sites. A similar mechanism of activation occurs when disulfides of cysteine are deaminated; thus cystine, cy-S—S-cy, or the mixed disulfide, cy-S—S-hcy, can be degraded to cysteine persulfide (as shown in the right column of Drawing 1) and cysteine(S)—S—CH₃ to CH₃—S—SH (left column). This degradation can be catalyzed by cystathionase or by other C—S lyases.

Transsulfuration pathway enzymes are frequently cited as a source of hydrogen sulfide. However, the enzymes of this pathway have been shown to be absent in the embryo (8), many cancers (partially reviewed in 9), and some virus infections (10) including HIV-AIDS (11). It is well-known that congenital defect in cystathionase has no adverse effects (12). Mice with knockout of cystathionase are healthy but require cyst(e)ine (13). Therefore, this pathway has to be discounted as a significant source of sulfane sulfur in most tissues.

The polyamine pathway. This pathway can generate sulfane sulfur in two different ways. After decarboxylation of S-adenosylmethionine and removal of the propylamine group, the methylthioadenosine undergoes phosphorylation releasing adenine and forming methylthioribose-1-phosphate. This is converted to 2-keto-4-methylthiobutyrate (KMTB) (14). This keto acid is the most avid amino group acceptor of all keto acids tested in transamination reactions (15-17) and provides a favorable keto acid for transaminating cysteine to mercaptopyuvate.

The KMTB formed in the polyamine pathway can give rise to sulfane sulfur by another route. It is known to be degraded in animal tissues to methylmercaptan (18-20) which enters the disulfide exchange system to give cysteine(S)—S—CH₃. As indicated above, this disulfide is degraded to CH₃—S—SH. This C—S lysis is catalyzed by a large number of pyridoxal phosphate-containing enzymes including amino acid transaminases (21), deaminases (21), decarboxylases (22), and others (21). The outer sulfur of the methyl persulfide is a sulfane sulfur which is immediately bound and transported by sulfane sulfur carriers such as rhodanese (5). Little is known about which C—S lyase may catalyze this reaction in various tissues. The removal of the sulfane sulfur atom from CH₃—S—SH leaves the mercaptan intact and the process can recycle.

The polyamine pathway appears to be involved as a major source of sulfane sulfur in animals as first revealed by the requirement for sources of sulfane sulfur in cells lacking MTAP. The pathway occurs in all organisms: microbes, animals, and plants; even some viruse genomes encode the enzymes of this pathway. In plants, the pathway generates the plant hormone ethylene and it is probably the source of the elemental sulfur which occurs in xylem as a defense againt fungus infection (23) and in the epicutical wax of both gymnosperms and angiosperms (24). In animals, the polyamine pathway salvages methionine and generates two products, polyamines and sulfane sulfur. Although polyamines are theorized to have functions based on their charge properties (e.g. binding to nucleic acids), attempts to reveal an essential function in animal cells have been largely unsuccessful (25). When bacterial cells were depleted in all the enzymes of the pathway in order to show a growth requirement, the cells multiplied without the pathway (26). Inhibitors of the pathway such as difluoromethylornithine can retard cell proliferation and this is frequently cited as evidence that polyamines are required without taking into account the other products of the pathway—salvaged methionine and sulfane sulfur. When all of these factors are taken into account, it appears likely that sulfane sulfur is the essential product of this pathway in animals.

### 4. Polyamine Pathway; Regulation, Macrophage Polarization, Methionine Dependence

a) The polyamine pathway is finely regulated. It has been repeatedly shown that this pathway is precisely correlated with stages of cell division in synchronously dividing cells. The three polyamines, putrescene, spermine and spermidine, undergo exquisitely-controlled increases in cells during each cell cyle (27). Three of the enzymes, ornithine decarboxylase (28-30), S-adenosylmethionine decarboxylase (28), and MTAP (31) each go through two peaks of activity during each cell cycle with one peak just before DNA synthesis (S phase) and one just before mitosis (M phase). Ornithine decarboxylase, the rate-controlling enzyme, has a extremely short half-life measured in minutes and it is degraded not by the usual ubiquitination process but by a special antizyme (28). These facts show that the polyamine pathway has critical and time-related functions in the process of cell division.
b) The polyamine pathway in macrophage polarization; the arginine dichotomy. Macrophages become “polarized” into two main types, M1 and M2. Macrophages of type M1 are the classical “activated” macrophages associated with inflammation, phagocytosis of foreign material, and cancer suppression. Macrophages of type M2 (frequently called “alternative”) are associated with tissue repair, cell proliferation, and cancer stimulation (extensively reviewed e.g. 32-33 and concisely summarized in 34). The polarization of macrophages to the two types is determined by the metabolism of arginine which can follow either of two pathways (Drawing 2). In M1 macrophages, arginine enters the nitric oxide pathway and gives rise to NO and citrulline. The NO has vasoactive and inflammatory functions and the citrulline is converted back to arginine. In M2 macrophages, arginine is cleaved by arginase to urea and ornithine. The ornithine enters the polyamine pathway and gives rise to polyamines and sulfane sulfur as decribed above. The generation of sulfane sulfur in M2 macrophages is consistent with their role in tissue repair and the role of sulfane sulfur in cell division. Macrophage polarization is determined by a complex system of cytokines and it is flexible since the cells can switch from one type to the other (35).
c) Methionine dependence of cancer. Briefly, in 1974 some evidence gave rise to the theory that cancer cells could not multiply in vitro when methionine was replaced by homocysteine whereas normal cells could multiply under these conditions (36). An inability of the methionine dependent cells to synthesize methionine was quickly ruled out since they all contained active methionine synthase. In 2000, Tang et al. tested 12 cell lines for the activity of methylthioadenosine phosphorylase (MTAP) and found almost exact correlation; six cell lines containing MTAP could grow with homocysteine (no methionine) but cells lacking MTAP required methionine (37). Since all of the cells could convert homocysteine to methionine, this is strong evidence that the cells are not “methionine dependent” but “dependent on some product derived from methionine via the polyamine pathway”. All of the evidence indicates that that product is sulfane sulfur.
d) Methionine restriction and longevity. In this field, dietary restriction of methionine (and absence of cysteine) has been found to increase the life span of rhodents, fruit flies, nematodes, and yeast (38). Other effects that have been demonstrated include decreased obesity, increased insulin sensitivity, and delayed progression of cancer in several rhodent models.
e) Countermeasures; Dietary sulfur restriction. Based on the above observations, dietary restriction of sulfur has been extensively studied and found to have anti-cancer effects as well as increasing longevity (39). In agreement with this, dietary sulfur restriction has been shown to redirect macrophages from type M2 (cancer-promoting) to type M1 (cancer-killing) (40). An alternative approach to dietary sulfur restriction is to block the polyamine pathway using enzyme inhibitors such as DFMO. Although these inhibitors are effective in vitro, they have produced little success and major toxicity in clinical trials. Another strategy has been to administer a recombinant enzyme, methioninase, which degrades methionine. Taken together, the evidence indicates that the benefits of methionine restriction are not attributable to limitation of methionine itself but to limitation of sulfane sulfur derived from methionine and its growth factor effect. This evidence also supports the concept that too much sulfane sulfur can be detrimental. The novel rationale of this invention is not to prevent the natural generation of sulfane sulfur but to capture and remove the excess that occurs in certain disease states. Patent application #69,974,552 is the first disclosure of the strategy of depleting sulfane sulfur from the body with binding agents.

### 5. Disease States Involving Macrophages, the Polyamine Pathway, and Sulfur

5a) Virus Infection. There is a close correlation between virus infection and the polyamine pathway. This is true for all viruses that infect bacteria, plants, and animals (exhaustively reviewed e.g. 41). Concomitant with the infection of cells by viruses there is a pronounced increase in the activity of the polyamine pathway. Most viruses trigger an increase in the host cell enzymes for the polyamine pathway (42) but some viruses carry their own complete set of genes for these enzymes—for example Chlorella viruses (43). In another example, the rice dwarf virus encodes a protein (Pns11) which specifically enhances S-adenosylmethionine synthase and accelerates the polyamine pathway (44). Given the extreme limitations on the size of the genome in viruses, the presence of these genes demonstrates the importance of a product of this pathway to virus replication. Conversely, when the pathway is inhibited with enzyme inhibitors (e.g. DFMO), there is a marked decrease in the viral load (numerous references, see 45).

In addition to being correlated to polyamine production, virus infection in animals is intimately related to the presence of macrophages. These defense cells are purposefully attracted to foreign agents such as invading viruses but, counterintuitively, M2 macrophages are believed to be preferred host cells for invasion by some viruses (46) and, since they are mobile, they can transport the viruses to remote areas of the body. For example, it has been shown that Ebola virus preferentially infects macrophages of type M2 (47)

In the case of the corona virus, Covid 19 (SARS CoV-2), macrophages are especially vulnerable because the they carry the transmembrane ACE receptor (angiotensin converting enzyme) which is the specific binding site for the virus (48). To add to the “perfect storm”, the macrophages associated with early corona virus invasion tend to be polarized to type M2 (49,50). It is likely that the sulfane sulfur generated by these M2 cells is required during the infection process of Covid 19 although the exact mechanism is not known. Virus infection of cells appears to increase the demand for sulfur amino acids (51). This is demonstrated for canine distemper virus cultivated in Vera cells (52).
5b. Cancer. It has been repeatedly shown that cancer is associated with increased activity of the polyamine pathway with increased levels of polyamines in the tissue, blood, and urine (53) to the extent that urinary polyamines has been said to be a “marker” of the cancer burden (54). At the same time, macrophages occupy considerable space in the cancer tissue and are associated with poorer prognosis (55). Tumor-associated macrohages are predominantly of type M2 (33) which produce polyamines. Interestingly, this may have a connection to theory that dogs can detect cancer by smell since polyamines have a pronounced pungent (fishy) odour—even to humans. In addition, the odour of polyamines has also been shown to attract insects: fruit flies and mosquitoes (56).
5c) Diet-induced fatty liver. In the 1930's-50's, there was intensive investigation of fatty liver induced in rats by dietary manipulation of sulfur-containing amino acids. Interest in this aspect of sulfur metabolism faded after 1960 but the subject has renewed interest in recent years because of the rising epidemic of fatty liver in humans (the world-wide prevalence being 25%). The voluminous early data on the relationship of dietary sulfur amino acids to fatty liver in experimental animals was reviewed in 2014 by this author and re-interpreted on the basis of the new knowledge on the role of sulfane sulfur (57). Briefly, the data are interpreted as follows; The enzymes of de novo lipid synthesis are controlled by persulfidation with high levels of persulfide inhibiting and low levels stimulating the process. Dietary methionine, which has been known to inhibit fatty liver since the 1920's, provides sulfane sulfur and slows lipid synthesis. Excess dietary cystine generates sulfite which binds and removes the sulfane sulfur and allows lipid synthsis to procede uncontrolled. In 2014, published data showed that the hydrogen sulfur/sulfane sulfur mixture could replace methionine in preventing fatty liver (58). This finding has been confirmed several times since 2014 as reviewed in (59). The hydrosulfide/So agent causes a decrease in both the mRNA and the enzyme proteins of fatty acid synthesis and, as well, an increase in the carnitine palmitoyl transferase (part of the lipid transport system). More recently, it has been shown that the sulfane sulfur precursor, dithiolthione, has the same protective effect against diet-induced fatty liver (60).

New evidence also confirms that the polyamine pathway in macrophages is involved in controlling fatty liver. Thus, mice with homozygous knock-out of arginase 2 develop severe fatty liver. The involvement of macrophages was shown by administering clodronate to deplete macrophages whereupon fatty liver was prevented and the activity of enzymes of lipid synthesis were decreased (61). Since the whole mice were Arg2−/−, the particular macrophage type involved were not identified with certainty.

A major factor in the current epidemic of fatty liver in humans is believed to be the high consumption of “high fructose corn syrup” (62). The causal relatiohsip is well-documented in experimental animals where dietary fructose causes a two-fold increase in the activity of fatty acid synthase (63). High-fructose corn syrup is manufactured industrially by hydrolysis of cornstarch (a glucose polymer) with amylase followed by isomerization of about half of the glucose to fructose by immobilized isomerase. The product is in liquid form with a small amount of water and it is not purified by crystallization. The relationship of this product to fatty liver seems incontrovertable but the mechanism by which this simple sugar product could induce fatty liver has been baffling. Many mechanisms have been proposed. However, there is a clear-cut and previously-unrecognized explanation involving sulfane sulfur removal as follows.

The industrial production of high fructose corn syrup involves two steps that could introduce sulfane sulfur binding agents relevant to this application—sulfite ion and cyanide ion (see “madehow.com/Volume-4/corn-syrup”). Sulfur dioxide is added to the corn starch in an early step and is likely to become attached to the sugars through an addition reaction involving carbonyl groups: R—C(O)H+HSO₃⁻→R—CH(OH)—SO₃. Later in the process, there are two treatments with activated charcoal which is known to contain significant amounts of cyanide ion which is formed during the pyrolysis. Cyanide ion also adds to carbonyl groups of sugars: RC(O)H+HCN→R—CH(OH)—CN.

Both of the addition products (sulfite and cyanide) are moderately stable and, since there is no step in the process to remove them, they may end up in the final product in significant amounts. After ingestion, they could break down slowly releasing sulfite or cyanide ions which would then bind tightly to the sulfane sulfur in the body and cause it to be excreted in the urine as outlined in Section 6c below. In ongoing continuous exposure, this would be expected to deplete the liver of rate-controlling sulfane sulfur thus increasing the rate of de novo fatty acid synthesis.

As an interesting aside, it might be pointed out that there are precedents for the accidental ingestion of cyanide. Thus, cyanide is ingested in cigarette smoke and in improperly processed cassava. The latter contains cyanogenic glycosides which are consumed in a diet that is already severely deficient in sulfur amino acids. In both instances, there is nerve pathology; “tobacco amblyopia” in cigarette smoking (64) and upper motor impairment (konzo and “tropical ataxic neuropathy” in cassava ingestion (65). Another example may be various forms of lathyrism (neuro-, osteo, angio-lathyrism) caused by ingestion of the grass pea (Lathyrus) which contains cyanide in the form of beta-aminopropionitrile (66). The diagnostic criterion for these disease is the detection of thiocyanate in the blood and urine (see Section 6c). Traditionally, these diseases have been attributed to inactivation of Vitamin B₁₂ and the unidentified role of this vitamin in nerve function (thought to involve methylation of myelin). But today, the new information on sulfane sulfur opens a new interpretation. Thus, chronic cyanide ingestion may deplete sulfane sulfur and the deficiency of the sulfane sulfur rather than Vitamin B₁₂ in nerve tissue may be the cause of the neurological defects.

### 6. Sulfane Sulfur Binding Agents.

### a) Inorganic Sulfites.

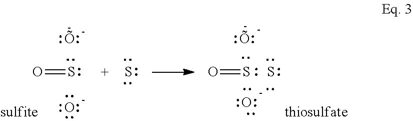

As used here, “sulfite” refers to the many inorganic forms of the ion; sulfite SO₃²⁻ (at pH>7), bisulfite HSO₃⁻ (at pH 3 to 7), or the dimers dithionite S₂O₄² and metabisulfite S₂O₅²⁻. They all occur in the form of a salt because the acid form is not stable. Sulfur dioxide gas reacts with water at pH above 3 to form sulfurous acid, H₂SO₃, whch has a pKa₂ of ˜7. At pH below pH 3, hydrosulfite degrades back to sulfur dioxide and water.

The binding of sulfane sulfur by sulfite ion is a natural process and the product, thiosulfate, is a natural excretory product of sulfur metabolism (67). The reaction is sufficiently robust that it can be used a reliable quantitative method for determining sulfane sulfur in biological materials. Sulfites are not toxic and are ingested by humans in many foods such as salads, wines, beers, and dried fruits. Good wines contain 50 to 100 mg of sulfite per litre (range 10 to 350 mg/1). It is possible that the well-known health benefits of the Mediterranean diet are due to the consumption of wines with high sulfite content.

Sulfite addition products. Besides the inorganic sulfites mentioned; sulfite can be administered as the sulfite addition product to aldehydes. These addition compounds are not very stable and release sulfite ion slowly (as described above). Other organic sulfite compounds include the sulfite derivatives of pararosaniline dyes of controversial structure.
b) Organic sulfinates are comparable in structure to inorganic sulfite except that an OH group of sulfite is replaced by an organic group. The two categories are similar in that the sulfinyl sulfur atom in both has a pair of unused electrons that can be used to bond sulfane sulfur (Eqs. 4,5). Cysteine sulfinate and hypotaurine are normal physiological chemicals and are intermediates in sulfur metabolism. Cavallini et al showed that hypotaurine combines with sulfane sulfur forming thiotaurine which is excreted in the urine (67). Other potentially useful organic sulfinates include thiourea dioxide (also called formamidine sulfinate) and hydroxymethane sulfinate.

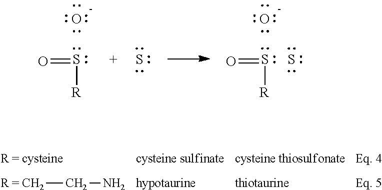

c) Nitriles. Cyanide ion is the classical sulfane sulfur-binding agent and is used in quantitative analysis.

:C:::N:⁻+:S:→:S:C:::N:⁻ Eq. 6

Hydrogen cyanide is well-known as a toxic agent and cannot be used directly. However, it reacts with aldehydes and ketones to give addition products called cyanohydrins: R—CH(O)+HCN→R—CH(OH)—CN. The reaction is reversible and the cyanohydrins slowly release cyanide when exposed to water. There are many cyanohydrins and, depending on the chemical structure, they release cyanide at different rates. Examples of slow cyanide release are cyanohydrins of mandelonitrile and glycolonitrile.
d) Phosphines. These tri-substituted phosphorus compounds have a pair of unused electrons that are strongly attractive to sulfane sulfur. The tri-hydrogen compound (phosphine itself) and low molecular weight derivatives are quite toxic and odorous. However, there are derivatives that are relatively odorless and used routinely in biochemsitry labs, e.g. tricarboxyethylphosphine (TCEP), and trihydroxypropylphosphine.

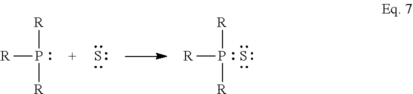

e) Nitrite Ion. This ion is traditionally added to foods as a preservative, for example in “corned beef”. It has no established toxiciy. It autoxidizes slowly to nitrate which is excreted in the urine. The nitrite ion binds sulfane sulfur in a complex mechanism to give a persulfonitrite (68).

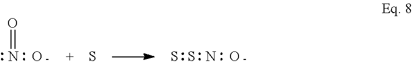

### 7. The Bell-Shaped Curve.

As with all regulatory agents, the dose-response profile for sulfane sulfur follows a bell-shaped curve with pathological effects resulting from too little or too much. That is demonstrated in the interpretation of the examples cited here. Too little failed to support in vitro proliferation of the lymphoma cells (L1210, P388) but too much in vivo appears to result in cancer. Too little is thought to allow unrestricted de novo fatty acid synthesis in the liver but too much appears to support virus infection. This patent application applies only to the instances of “too much” where removal of the excess may correct the pathology. Another probable example of “too much” (not covered here in detail because of lack of evidence) is the role of sulfane sulfur in connective tissue. Congenital defects in enzymes of sulfur metabolism cause severe hyperhomocysteinemia and/or hypermethioninemia resulting in connective tissue pathology and atherosclerosis. A relationship to sulfane sulfur generation has not been documented but it seems likely that there is a dysregulation in the enzymes of collagen synthesis similar to that proposed for the enzymes of fatty acid synthesis in fatty liver disease. The scope of sulfane sulfur in regulation will probably continue to expand—already including cell proliferation, nerve function, liver metabolism, virus infection, macrophage function, and probably connective tissue synthesis.

### EXAMPLES AND SIGNIFICANCE OF THIS INVENTION

### Example 1. Ingesting Sulfite Blocks Infection by the Rhinovirus of the Common Cold

Whenever, this inventor has the first symptoms of the common cold, he ingests K. metabisulfite (the agent added to wines) in quantities of 250 mg every 4 hours for 24 hours and the symptoms clear without progressing to a full cold. It appears to be important that this be started within 48 hours of first appearance of the symptoms although data is lacking on using the agent later in the course of the infection. The stated quantities are effective but might be refined. The total quantity in the above example is equivalent to drinking 4 bottles of some wines or ingesting 500 gm of certain dried fruits. Application of this example to Covid 19 would be a first TREATMENT for this disease.

### Example 2. Applicability to Plant Pathogens

Although tests have not been done, the applicability of this invention to diseases caused by plant viruses has some urgency because plant pandemics are equally as possible as animal pandemics and have the potential to cause famine. The spotted tomato wilt virus is said to be “one of the most economically devastating plant viruses in the world” (Wikipedia). Plants have the same polyamine pathway as animals and it is greatly increased during virus infection. Indeed, the Chlorella virus that encodes all the enzymes of the pathway is a parasite of green algae. In treating virus infection of plants (as opposed to animals), the cyanohydrins and phosphines may be more applicable than in treating disease in animals.

### Example 4. Cancer

Sulfane sulfur was discovered because cells lacking it could not multiply in vitro and there is now a working hypothesis that overabundance of sulfane sulfur promotes cellular hyperproliferation and cancer. There are already clinical trials on strategies that would deplete sulfane sufur (dietary sulfur restriction, recombinant methioninase to destroy methionine in vivo, and inhibitors of the polyamine pathway). In this patent application, a novel strategy is described—the binding and removing pre-formed sulfane sulfur by specific binding agents.

### REFERENCES

1. Toohey J I, Cline M J, Alkylthiolation. Evidence fo involvement in cell division. Biochem. Biophys. Res. Commun. 70, 1275-1282 (1976).
2. Smith D J, Maggio E T, Kenyon G L, Simple alkanethiol groups for temporary blocking of sulfhydryl groups of enzymes. Biochemistry, 14, 766-771 (1975).
3. Toohey J I, Persulfide sulfur is a growth factor for cells defective in sulfur metabolism. Biochem. Cell Biol. 64, 758-765 (1986).
4. Toohey J I, Methylthioadenosine nucleoside phosphorylase deficiency in methylthio-dependent cancer cells. Biochem. Biophys. Res. Commun. 83, 27-35 (1978).
5. Toohey J I, Cooper A J L., Thiosulfoxide (sulfane) sulfur: new chemistry and new regulatory functons in biology. Molecules 19, 12789-12813 (2014). doi 10.3390/molecules190812789
6. Fukuto J M, et al., Biological hydropersulfides and related polysulfides—a new concept and perspective in redox biology. FEBS Lett. 592, 2140-2152 (2018).
7. Toohey J I, Macrophages and methylthiogroups in lymphocyte proliferation. J. Supramol. Struct. Cell. Biochem. 17, 11-25 (1981).
8. Sturman J A, Gaull G, Riha N C, Absence of cystathionase in human fetal liver: Is cystine essential? Science 169, 74-75 (1970). Pascal T A, Gillam B M, Gaull G E, Cystathionase: Immunochemical evidence for absence from human fetal liver. Pediat. Res. 6, 773-778 (1972). Sunkara, Gaull G, Sturman J A, Raiha N C, Development of mammalian sulfur metabolism; absence of cystathionase in human fetal tissues. Pediat Res 6, 538-547 (1972). Zlotkin S H, Anderson G H, The development of cystathionase activity during the first year of life. Pediat. Res. 16, 65-68 (1982).
9. Glode L M, Epstein A, Smith C G, Reduced γ-cystathionase protein content in human malignant leukemia cell lines as measured by immuno asay with monclonal antibody. Cancer Res. 41, 2249-2254 (1981). Klein C E, Roberts B, Holcenberg J, Glode L M, Cystathionine metabolism in neuroblastoma. Cancer 62, 291-298 (1987).
10. Livingston D M et al., Accumulation of cystine auxotrophic thymocytes accompanying type C viral leukemogenesis in the mouse. Cell 7, 41-47 (1976).
11. Martin J A, Sastre J, de la Asuncion J G, Pellardo F V, Vita J, Hepatic γ-cystathionase deficiency in patients with AIDS. J. Am. Med. Ass. 285, 1444-5 (2001).
12. Mudd S H, Levy H I, Kraus S, Metabolic basis of inherited disease pp. 2007-2056 (2000). Levy H L, Mudd S H, Uhlendorf B W, Madigan P M, Cystathioninuria and homocystinuria. Clin. Chim. Acta. 58, 51-59 (1975).
13. Ishii I, Akahoshi N, Yamada S, Nakano S, Izumi T, Suematsu M, Cystathionine γ-lyase-deficient mice require dietary cystine to protect against acute lethal mypopathy and oxidative injury, J. Biol. Chem. 285, 26358-26368 (2010).
14. Toohey J I, Ketomethylthiobutyric acid formation from methylthioadenosine: A diffusion assay. Arch. Biochem. Biophys. 223, 533-542 (1983). Backlund P S, Chang C, Smith R A, Identification of 2-keto-4-methylthiobutyrate as an intermediate compound in methionine synthesis form 5′-methylthioadenosine. J. Biol. Chem. 257, 4196-4202 (1982).
15. Cooper A J L, Meister A, Isolation and proprties of a new glutamine transaminase from rat kidney. J. Biol. Chem. 249, 2554-2561 (1974).
16. van Leuven F, Glutamine transaminase from brain tissue. Eur. J. Biochem. 65, 271-274 (1976).
17. Ricci G, Nardini M, Federici G, Cavallini D, The transamination of L-cystathionine, L-cystine and related compounds by a bovine kidney transaminase. Eur. J. Biochem. 157, 57-63 (1986).
18. Blom H J, et al. Methanethiol and dimethylsulfide from 3-methylthiopropionate in human and rat hepatocytes. Biochim. Biophys. Acta 972, 131-136 (1988).
19. Steele R D, Benevenga N J, The metabolism of 3-methylthiopropionate in rat liver homogenates. J. Biol Chem. 254, 8885-8890 (1979).
20. Blom H J, van den Elzen J P, Yap S H, Tangerman A, Methane thiol and dimethylsulfide formation from 3-methylthiopropionate in human and rat hepatocytes. Biochim. Biophys. Acta. 972, 131-136 (1988). Scislowski P W, Bremer J, van Thienen W I, Davis, E J, Heart mitochondria metabolize 3-methylthiopropionate to CO₂ and methanethiol. Arch. Biochem. Biophys. 273, 602-605 (1989)
21. Cooper A J L, Pinto J, Cysteine S-conjugate β-lyases. Amino Acids. 30, 1-15 (2006)
22. Buckberry L D, Patel R, Hollingworth L, Teesdale-Spittle P H. Cysteine conjugate β-lyase activity of amino acid decarboxylases. Biochem. Soc. Trans. 26, 269S (1998).
23. Cooper R N, Williams J S, Elemental sulfur as an inducible antifungal substance in plant defense. J. Exp. Bot. 55, 1947-1955 (2004). Kylin K, Elemental sulphur (S8) in higher plants—biogenic or andropogenic origin. Experientia 50, 80-85 (1994).
24. Zhao S, et al. A viral protein promotes host SAMS1 activity and ethylene production for the benefit of virus infection. eLife 2017:e27529. doi 10.7554/3Life. 27529
25. Miller-Fleming L, et al. Remaining mysteries of molecular biology: The role of polyamines in the cell. J. Mol. Biol. 427, 3389-3406 (2015).
26. Chattapadhyay M K, et al., Polyamines are not required for aerobic growth of Escherichia coli: preparation of a strain with deletions in all of the genes for polyamine biosynthesis. J. Bact. doi 10.1128/JB.00381-09
27. Kusunoki S, Yasumasu I, Cyclic change in polyamine concentrations in sea urchin eggs related with cleavage cycle. Biochem. Biophys. Res. Commun. 68, 881-885 (1976).
28. Heby O, Gray J W, Lindl P A, Marton L J, Wilson C B, Changes in L-ornithine decarboxylase activity during the cell cycle. Biochem. Biophys. Res. Commun. 71, 99-105 (1976).
29. McCann P P, Tardif C, Mamont P S, Schuber F, Biphasic induction of ornithine decarboxylase and putrescine levels in growing HTC cells. Biochem Biophys. Res. Commun. 64, 336-341 (1975).
30. Fredlund J O, Johansson M C, Dahlberg E, Oredsson S M, Ornithine decarboxylase and S-adenosylmethionine decarboxylase expression during the cell cycle of Chinese hamster ovary cells. Exp. Cell Res. 216, 86-92 (1995).
31. Sunkara P S, Chang C C, Lachman P J, Cell proliferation and cell cycle-dependent changes in the methylthioadenosine phosphorylase activity in mamalian cells. Biochem. Biophys. Res. Commun. 127, 546-551 (1985).
32. Sica A, Montovani A. Macrophage polarization in tumor progression. Seminars Cancer Biol. 18, 349-355 (2008) doi 10.1016/j.semcancer.2008.03.004
33. Hardbower D M, etal. Ornithine decarboxylase regulates M1 macrophage activation and mucosal inflammation via histone modification. Proc. Natl. Acad. Sci. 114, E751-E760 (2017).doi 10.1073/pnas.161498114
34. Levy K. M1 means kill; M2 means heal. J. Immunol. 199, 2191-93 (2017) doi 10.4049/j.immunol.1701135
35. Sica A, Mantovani A, Macrophage plasticity and polarization: in vivo veritas J. din. Invest. 122, 787-795 (2012)).
36. Chaturvedi S, Hoffman R M, Bertino J R, Exploiting methionine restriction for cancer treatment. Biochem. Pharmacol. 154, 170-173 (2018).
37. Tang B, Li Y, Kruger W D, Defects in methylthioadenosine phosphorylase are associated with but not responsible for methionine-dependent tumor cell growth. Cancer Res. 60, 5543-5547 (2000).
38. Ables G P, Johnson J E, Pleiotropic responses to methionine restriction. Exp. Gerontol. 94, 83-88 (2017)
39. Hoffman R M, Clinical studies of methionine-restricted diets for cancer patients. Methods Mol. Biol. 1866, 95-105 (2019).
40. Orillion A, Damayanti M P, et al., Dietary protein restriction reprograms tumor-associated macrophages and enhances immunotherapy. Clin. Cancer Res. 24, 6383-6395 (2018).
41. Firpo M R, Mounce B C, Diverse functions of polyamines in virus infection. Biomolecules 10, 628 (2020) doi 10.3390/biom10040628
42. Mounce B C, Olsen M E, et al. Polyamines and their role in virus infection. Microbiol. Mol. Biol. Rev. 81, pii: 00029-17 (2017) doi 10.1128/MMBR.00019-17
43. Morehead T A, Gurnon J R, et al., Ornithine decarboxylase encoded by chlorella virus PBCV-1.Virology, 301, 165-175 (2002). Baumann S, Sander A, Gurnon J R, et al. Chlorella viruses contain genes encoding a complete polyamine biosynthetic pathway. Virology, 360, 209-217 (2007).
44. Zhao S, et al. A viral protein promotes host SAMS1 activity and ethylene production for the benefit of virus infection. eLife 2017; 6:e27529 doi 10.7554/eLife.27529
45. Mounce B C, et al. Inhibition of polyamine biosynthesis is a broad spectrum strategy against RNA viruses. J. Virol. 90, 9683-92 (2016) doi 10.1128/JVI.01347-16 Tyms A S, Williamson J D, Inhibitors of polyamine biosynthesis block human cytomegalo virus replication. Nature 297, 690-691 (1982). doi 10.038/297690a0
46. Nikitina E, Monocytes and macrophages as viral targets and reservoirs 10.3390/ijms19092821
47. Olsen M E, Filone C M, et al., Polyamines and hypusination are required for Ebola virus gene expression and replication. MBio 7, e00882-16. doi 10.1128/mBio.00882-16
48. Abassi Z, et al., The lung macrophage in SARS-CoV-2 infection: Friend or foe? 10.3389/fimmu.2020.01312
49. Page C, et al., Induction of alternatively activated macrophages in SARS Coronavirus infection J. Virol., 86, 13334-49 (2012) doi 10.1128/jvi01689-12
50. Moin A S M, et al. Pro-fibrotic M2 macrophage markers may increase the risk for Covid 19 in type 2 diabetes with obesity. Metabolism Clin. Exper. 112, 154374 Nov. 1 (2020). 10.1016/j.metabol.2020.154374
51. Koziorowski J., et al., Methionine dependence of virus-infected cells. Exp. Cell Res. 190, 250-253 (1990).
52. Hirayama N, Requirement for methionine for replication of canine distemper virus in Vero cells. J. Gen. Virol. 66, 149-157 (1985).
53. Casero R A, Stewart T M, Pegg A E, Polyamine metabolism and cancer: treatments, challenges and opportunities. Nat. Rev. Cancer 18, 681-695 (2018).
54. Durie B G, Salmon Se, Russell D A, Polyamines as markers of response and disease activity in cancer chemotherapy. Cancer Res. 37, 214-221 (1977).
55. Bachrach U, Polyamines and cancer: minireview. Amino Acids 26: 307-309 (2004). Nielsen S R, Schmid M L, Macrophages as key drivers of cancer progression and metastasis. Mediators Inflamm. 2017. 9624760 doi 10.1155/2017/962470
56. Akasaka N, et al. Polyamines in brown rice vinegar function as potent attractants for the spotted wing drosophila. J. Biosci. Bioeng. 123, 78-83 (2017)
57. Toohey J I. Sulfur amino acids in diet-induced fatty liver: a new perspective based on recent findings, Molecules 19, 8334-49 (2014) doi 10.3390/molecules19068334
58. Luo Z L, et al. Effects of treatment with hydrogen sulfide on methionine-choline deficient diet-induced non-alcoholic steatohepatitis in rats. J. Gastroenterol. Hepatol. 29, 215-221 (2014).
59. Wu D, et al. Hydrogen sulfide as a novel regulatory factor in health and disease. Oxid. Med. Cell. Longev. 2019. 3831713 doi 10.1155/2019/3831713 Wu D, et al. Exogenous hydrogen sulfide mitigates the fatty liver in obese mice through impairing lipid metabolism and antioxidant potential. Med. Gas Res. January (2015) doi 10.1186/s.13618-014-0022-y
60. Zhao C, et al., Slow release H₂S donor anethole dithiolethione protects liver from lipotoxicity by improving fatty acid metabolism. Front. Pharmacol. 11.549377 (2020) doi 10.3388/fpharm.22020.549377
61. Navarro L A, et al., Arginase 2 deficiency results in spontaneous steatohepatitis: a novel link between innate immune activation and hepatic de novo lipogenesis. J. Hepatol. 62, 412-420 (2015). doi 10.1016/j.jhep.2014.09.015
62. Jensen T. et al. Fructose and sugar: A major mediator of non-alcoholic fatty liver disease. J. Hepatol. 68, 1063-75 (2018) doi 10.1016/j.jhep.2018.08.004
63. Jegatheesan P, deBrandt J P, Citrulline and non-essential amino acids prevent fructose-induced nonalcoholic fatty liver in rats. J. Nutr. 145, 2273-79 (2015).
64. Matthews D M, Wilson J, Cobalamins and cyanide metabolism in neurological diseases. pp 115-136 in The Cobalamins: A Glaxo symposium ed. Arnstein H R V, Wrighton R J. Williams & Wilkin, Baltimore 1971
65. Kashala-Abotnes A, et al. Konzo: A distinct neurological disease associated with food (cassava) cyanogenic poisoning. Brain Res. Bull. 145, 87-91 (2019) doi 10.1016/j.brainresbull.2018.07.001
66. Llorens J., et al., A new unifying hypothesis for lathyrism, konzo, and tropical ataxic neuropahty: nitriles are the causative agents. Food Chem. Toxicol. 49, 563-570 (2011). doi 10.1016/j.fct.2010.06.005
67. Cavallini D, deMarcoC, Mondovi B, Chromatographic evidence on the occurrence of thiotaurine in the urine of rats fed with cystine. J. Biol. Chem. 234,854-857 (1959).
68. Cortese-Krott M M, et al. On the chemical biology of the nitrite/sulfide interaction. Nitric Oxide, 46, 14-24 (2015). 1.1016/j.niox.2014.12.009

What is claimed is:

1. A method of binding, inactivating, or removing disease-related excess or above-normal amounts of sulfane sulfur from an organism affected by a disease state that involves excess or above-normal amounts of sulfane sulfur which comprises the application of an agent that binds sulfane sulfur via a chemical reaction.

2. The method of claim 1 in which the organism is an animal, the disease state is a virus infection, and the sulfane sulfur-binding agent is an inorganic sulfite, an organic sulfinate, a cyanohydrin, or a phosphine.

3. The method of claim 2 in which the virus infection is a corona virus in a human.

4. The method of claim 3 in which the corona virus is Covid 19.

5. The method of claim 1 in which the organism is a plant, the disease state is a virus infection, and the sulfane sulfur-binding agent is an inorganic sulfite, an organic sulfinate, cyanide ion, a cyanohydrin, a phosphine, or a nitrite.

6. The method of claim 1 in which the organism is an animal and the disease state is cancer.
